(19) United States
(12) Patent Application Publication
(10) Pub. No.: US 2021/0378958 A1
(43) Pub. Date: Dec. 9, 2021

(54) Emulsions and extended release formulations including cannabis-derived compounds and related methods

(71) Applicant: Jeffrey Alan McKinney, Half Moon Bay, CA (US)

(72) Inventor: Jeffrey Alan McKinney, Half Moon Bay, CA (US)

(21) Appl. No.: 17/300,337

(22) Filed: May 14, 2021

Related U.S. Application Data
(60) Provisional application No. 63/102,261, filed on Jun. 4, 2020.

(51) Int. Cl. A61K 9/107; A61K 45/06; A61K 47/12

(52) U.S. Cl. CPC A61K 9/107; A61K 47/12; A61K 45/06

(57) ABSTRACT

The present invention generally relates to emulsions and extended release formulations including cannabis-derived compounds and related methods. It more specifically relates to emulsion-based and extended release formulations. In one aspect, the present invention is directed to an emulsion. The emulsion includes a compound derived from cannabis and an active ingredient. In another aspect, the present invention is directed to an extended release formulation. The formulation includes a compound derived from cannabis and an active ingredient.

### FIELD OF THE INVENTION

The present invention generally relates to emulsions and extended release formulations including cannabis-derived compounds and related methods.

### BACKGROUND OF THE INVENTION

There have been reports of emulsion-based and extended release formulations. For instance, U.S. Pat. No. 10,251,837, entitled “Compositions for nanoemulsion delivery systems” allegedly reports the following: “The present disclosure provides a pharmaceutically acceptable, stable, and optically clear oil-in-water nanoemulsions (intensity-averaged diameter<100 nm) with an oil phase comprising>10% w/v of long chain triglyceride, total surfactant and cosurfactant concentration less than that of oil phase and without the use of alcohol as cosolvent in the aqueous phase. The nanoemulsions of this disclosure have extremely favorable particle size distribution, optical clarity, and product stability against Ostwald ripening with high levels of oil concentrations. Poorly water soluble, therapeutically active agents and others can be incorporated in the nanoemulsion systems to improve their solubility/stability in aqueous medium or to enhance their delivery for use in pharmaceutical, food, cosmetic, and other applications by oral, intravenous, subcutaneous, intra muscular, inhalation, nasal, topical, ocular, and transdermal routes.” Abstract.

U.S. Pat. No. 10,105,330, entitled “Extended, delayed and immediate release formulation method of manufacturing and use thereof” allegedly reports the following: “Compositions for reducing the frequency of urination and methods of manufacturing the compositions are disclosed. The compositions comprises a first component having an immediate-release subcomponent and an extended-release subcomponent, wherein the first component is formulated to release the subcomponents immediately after administration; and a second component comprising an immediate-release subcomponent and an extended-release subcomponent, wherein the second component is formulated for a delayed-release of the subcomponents.” Abstract.

Despite the reports, there is still a need in the art for novel formulations and related methods.

### SUMMARY OF THE INVENTION

In one aspect, the present invention is directed to an emulsion. The emulsion includes a compound derived from cannabis and an active ingredient.

In another aspect, the present invention is directed to an extended release formulation. The formulation includes a compound derived from cannabis and an active ingredient.

### DETAILED DESCRIPTION OF THE INVENTION

“Active ingredient” refers to a compound or mixture that is used to achieve a particular biological, physiological or therapeutic outcome. Nonlimiting examples of such compounds include: CBD (cannabidiol); THC (tetrahydrocannabinol); and mixtures thereof.

“Compound derived from cannabis” refers to an isolated chemical compound that naturally occurs in a cannabis plant.

“Acid derived from cannabis” refers to an isolated chemical compound that includes a carboxylic acid and which naturally occurs in a cannabis plant. Nonlimiting examples of such compounds include: palmitic acid; stearic acid; oleic acid; vaccenic acid; linoleic acid; g-linoleic acid; arachidic acid; octadecatetraenoic acid; and eicosenoic acid.

“Oil derived from cannabis” refers to a viscous liquid that results from the processing of a cannabis plant. The oil includes one or more compounds derived from cannabis, including, without limitation, nonacosane, monoterpenes, sesquiterpenes and other terpenoid compounds (e.g., cannabis flower essential oil). The one or more compounds derived from cannabis further include, without limitation, myrcene, α-pinene, β-pinene, delta-3-carene, limonene, β-pellandrene, cis-ocimene, α-terpinolene, α-bergamotene, trans-caryophyllene, α-humulene, β-selinene, Selina-3,7(11)-diene, and caryophyllene oxide.

“Oil” refers to a viscous liquid. Nonlimiting examples of oil include: vitamin E acetate; olive oil; walnut oil; avocado oil; grapeseed oil; coconut oil; sesame oil; sunflower oil; soybean oil; and sweet almond oil.

“Surfactant” refers to a compound that lowers the surface tension between two liquids, between a gas and a liquid, or between a liquid and a solid. Nonlimiting examples of surfactants include: Tween-20; coco glucoside; decyl glucoside; lauryl glucoside; disodium laureth sulfosuccinate; coco betaine; sodium coco sulfate; Plantopon SF; soap nuts; liquid yucca extract; shikakai powder; soapwart.

Formulations of the present invention include compounds and compositions derived from cannabis. Nonlimiting examples of such compounds and compositions include: palmitic acid; stearic acid; oleic acid; vaccenic acid; linoleic acid; g-linoleic acid; arachidic acid; octadecatetraenoic acid; eicosenoic acid; oil, including one or more compounds derived from cannabis such as nonacosane, monoterpenes, sesquiterpenes and other terpenoid compounds (e.g., cannabis flower essential oil).

In one aspect, the present invention is directed to an emulsion including a compound derived from cannabis and an active ingredient. In another aspect, the present invention is directed to an emulsion including an acid derived from cannabis and an active ingredient. In another aspect the present invention is directed to an emulsion including an oil derived from cannabis and an active ingredient. In another aspect the present invention is directed to an emulsion including an acid derived from cannabis, an oil derived from cannabis and an active ingredient.

In still another aspect, the present invention is directed to an emulsion including an active ingredient, an oil, water and a surfactant. The following are nonlimiting examples of those three components:

1. An active ingredient, oil, water and one or more compounds selected from the following: palmitic acid; stearic acid; oleic acid; vaccenic acid; linoleic acid; g-linoleic acid; arachidic acid; octadecatetraenoic acid; eicosenoic acid.

2. An active ingredient, water, surfactant and oil derived from cannabis.

3. An active ingredient, an oil derived from cannabis, water and one or more compounds selected from the following: palmitic acid; stearic acid; oleic acid; vaccenic acid; linoleic acid; g-linoleic acid; arachidic acid; octadecatetraenoic acid; eicosenoic acid.

In another aspect, the present invention is directed to an extended release formulation including a compound derived from cannabis and an active ingredient. In another aspect, the present invention is directed to an extended release formulation including an acid derived from cannabis and an active ingredient.

In still another aspect, the extended release formulation of the present invention includes an active ingredient, a water-soluble polymer and a water-insoluble fatty acid. The following is a nonlimiting example of these components: an active ingredient, a water-soluble polymer and one or more compounds selected from the following: palmitic acid; stearic acid; oleic acid; vaccenic acid; linoleic acid; g-linoleic acid; arachidic acid; octadecatetraenoic acid; eicosenoic acid.

The following references are incorporated-by-reference into this document for all purposes:

Desai et al. “Fatty acid and water-soluble polymer-based controlled release drug delivery system” Journal of Pharmaceutical Sciences, 29 Nov. 2010, 100(5):1900-1912.

Gulluni et al. “Cannabis Essential Oil: A Preliminary Study for the Evaluation of the Brain Effects” Evidence-Based Complementary and Alternative Medicine, Volume 2018, Article ID 1709182, 11 pages.

Nakano et al. “Development of a Novel Nanoemulsion Formulation to Improve Intestinal Absorption of Cannabidiol” Med Cannabis Cannabinoids 2019;2;35-42.

Coggins, “24/7 Relief: Sustained-Release Cannabis Formulations” CRx Magazine, Vol. 1, No. 1, p. 34.

Molleken and Theimer 1997, “Survey of minor fatty acids in Cannabis sativa L. fruits of various origins” Journal of the International Hemp Association 4(1): 13-17.

“Cannabis flower essential oil”, Wikipedia, https://en.wikipedia.org/wiki/Cannabis_flower_essential_oil.

“The Art and Science of Cannabis Beverages”, https://www.newcannabisventures.com/wp-content/uploads/white_paper_ascb_v9.pdf.

1. An emulsion including an active ingredient, an oil, water and one or more compounds selected from the following: palmitic acid; stearic acid; oleic acid; vaccenic acid; linoleic acid; g-linoleic acid; arachidic acid; octadecatetraenoic acid; eicosenoic acid.

2. An extended release formulation including an active ingredient, a water-soluble polymer and one or more compounds selected from the following: palmitic acid; stearic acid; oleic acid; vaccenic acid; linoleic acid; g-linoleic acid; arachidic acid; octadecatetraenoic acid; eicosenoic acid.
